(19) United States
(12) Patent Application Publication
(10) Pub. No.: US 2019/0390193 A1
(43) Pub. Date: Dec. 26, 2019

(54) Reversible method for sustainable human cognitive enhancement

(71) Applicant: John Lawrence Mee, Stuart, FL (US)

(72) Inventor: John Lawrence Mee, Stuart, FL (US)

(21) Appl. No.: 16/016,562

(22) Filed: Jun. 23, 2018

(51) Int. Cl. C12N 15/11; C12N 9/22; A61P 25/00

(52) U.S. Cl. CPC C12N 15/11; C12N 2310/20; A61P 25/00; C12N 9/22

(57) ABSTRACT

A reversible method for general-purpose cognitive enhancement in mentally healthy adults comprising administering an RNA-editing ribonuclease complexed with a single guide RNA to lower the population of 5-hydroxytryptamine 2A receptors in the brain.

### APPLICATIONS CITED

Co-pending application Ser. No. 15/970,037, Method for sustainable human cognitive enhancement, and co-pending application Ser. No. 15/986,222, Adjustable method for sustainable human cognitive enhancement.

### FIELD OF THE INVENTION

The present invention relates generally to human genetic engineering, and more particularly to the application of transcriptome engineering methods and techniques to expand human cognitive capacity.

### BACKGROUND OF THE INVENTION

In the development of genetic engineering methods for improving human cognitive performance, there may be applications where it is desirable for cognitive changes to be reversible, such as in research and testing phases. Additionally, a reversible edit can enable candidates for genetic cognitive upgrades to acclimate themselves to enhanced cognitive states prior to receiving a permanent DNA edit.

### SUMMARY OF THE INVENTION

It is a principle object of the present invention to provide a genetic cognitive enhancer which delivers reversible higher states of awareness, concentration, focus, clarity, mental acuity, mindfulness and creativity.

It is a specific object of the invention to provide a safe and effective genetic cognitive enhancer which delivers the aforementioned results from a single, one-time application.

It is a final object of the invention to provide a genetic cognitive enhancer which does not affect the germline.

The present invention provides a method of achieving reversible, general-purpose cognitive enhancement in mentally-healthy adults comprising administering an RNA-editing ribonuclease complexed with a single guide RNA to lower the population of 5-hydroxytryptamine 2A receptors in the brain

Reducing a neuron's 5-hydroxytryptamine 2A receptor population raises its electrical resistance, thereby lowering its electrical conductivity and excitability. Higher electrical resistance in neurons decreases brain current density and attenuates brainwave activity. Diminished brainwave activity has been scientifically correlated with higher states of awareness, concentration, focus, creativity and mental acuity.

One aspect of the present invention provides a catalytically-active RNA-editing ribonuclease complexed with a single guide RNA to lower the population of 5-hydroxytryptamine 2A receptors in the brain by altering RNA nucleotides to repress translation of gene HTR2A into cellular proteins in CNS neurons.

Another aspect provides a catalytically-inactive RNA-editing ribonuclease complexed with a single guide RNA to lower the population of 5-hydroxytryptamine 2A receptors in the brain by binding to RNA nucleotides to repress translation of gene HTR2A into cellular proteins in CNS neurons.

Another aspect provides a catalytically-inactive RNA-editing ribonuclease complexed with a single guide RNA and a deaminase enzyme to lower the population of 5-hydroxytryptamine 2A receptors in the brain by causing RNA nucleobase substitutions which result in translational interference of gene HTR2A in CNS neurons.

Yet another aspect of the invention provides an RNA-editing ribonuclease complexed with an RNA-expression inhibiting nucleotide and a single guide RNA to lower the population of 5-hydroxytryptamine 2A receptors in the brain by altering RNA nucleotides to cause translational interference of gene HTR2A in CNS neurons.

Still another aspect of the invention provides a catalytically-active nuclease complexed with a single guide RNA to lower the population of 5-hydroxytryptamine 2A receptors in the brain by altering nucleotides in micro-RNA biogenesis sites to repress the expression of gene HTR2A in CNS neurons.

A further aspect provides a single guide RNA which transfects neurons.

Another aspect provides a single guide RNA which navigates the RNA-editing ribonuclease to the RNA for gene HTR2A.

A further aspect provides a single guide RNA which navigates the RNA-editing ribonuclease to micro-RNA biogenesis sites for the RNA for gene HTR2A.

Another aspect provides a method of calculating dosages for genetic cognitive enhancements.

A further aspect provides a psychological screening method for determining suitable candidates for genetic cognitive enhancement.

### DESCRIPTION OF THE DRAWINGS

FIG. 1 is an illustration of a neurowave flowing through a series of neurons

FIG. 2 is an illustration comparing neurons in series with transistors in series

FIG. 3 is a graph depicting a typical neuron's pulse rise time

FIG. 4 is an illustration of neurowave voltage and frequency

FIG. 5 is a diagram explaining neuron electrodynamics using a resistor-capacitor network example

FIG. 6 is a detailed illustration of the electrical characteristic of a neurowave

FIG. 7A is the first page of a flowchart diagram illustrating a method for achieving reversible human cognitive enhancement using genetic engineering

FIG. 7B is the second page of a flowchart diagram illustrating a method for achieving reversible human cognitive enhancement using genetic engineering

### DETAILED DESCRIPTION

### I. Definitions

Neurowaves: Brainwaves are composed of millions of tiny, cellular-level electromagnetic waves which travel through neurons. This application refers to these neuron-level electromagnetic waves as “neurowaves.”

### II. Overview

### 1. Brain Currents

A moving electrical current generates an electromagnetic wave (per Ampere's Law). Flowing electrons in the brain generate brainwaves. When the flowing electrons slow down, so does brainwave activity.

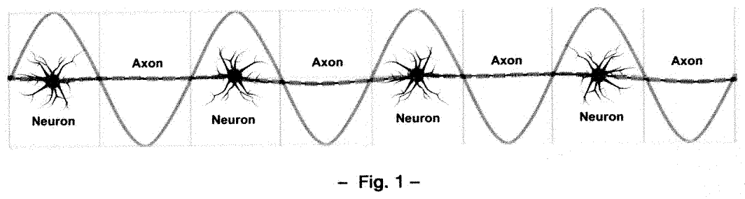

FIG. 1 shows a neurowave traveling through a series of neurons. Every neurowave has a corresponding flow of electrical current which runs through neurons in the brain.

Brain currents flow through neurons at different rates, depending on the neuron's physical properties. Neurons which have higher electrical resistance impede the flow of current, while neurons with lower resistance conduct current more readily.

When the flow of a brain current is impeded, its associated brainwave slows down. Slower brainwaves exhibit lower overall activity per second.

### 2. Neuron Electrodynamics

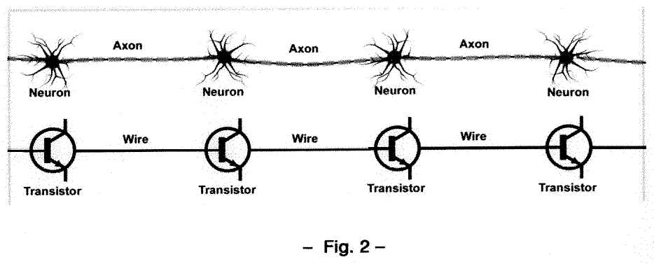

As shown in FIG. 2, the axon filaments connecting neurons resemble wires connecting transistors in a series. From a functional perspective, a neuron is a switching mechanism for electrical impulses, much like a transistor. Computers are made of transistors connected by wires, while brains are made of neurons connected by axons. In a computer, transistors act as logical switches which send electrical pulses along conducting wires. In the brain, neurons act as logical switches which send electrical pulses along interconnecting axons.

Research at Yale and Stanford has shown that flowing electrons in the brain's neural networks are accompanied by tiny electromagnetic waves typically measuring 55 millivolts and 5 nanoamperes. This relatively large voltage compared to the small amount of current is necessary to overcome the resistance of the brain's electro-chemical circuits, which is very high compared to ideal conductors like copper or gold.

Brainwave frequencies, conventionally expressed as a number between 1 to 40 Hertz, measure the average number of neuron conversations per second. When it takes longer for one neuron to talk to the next one, there are fewer neuron conversations in any given unit of time, and brainwave activity diminishes.

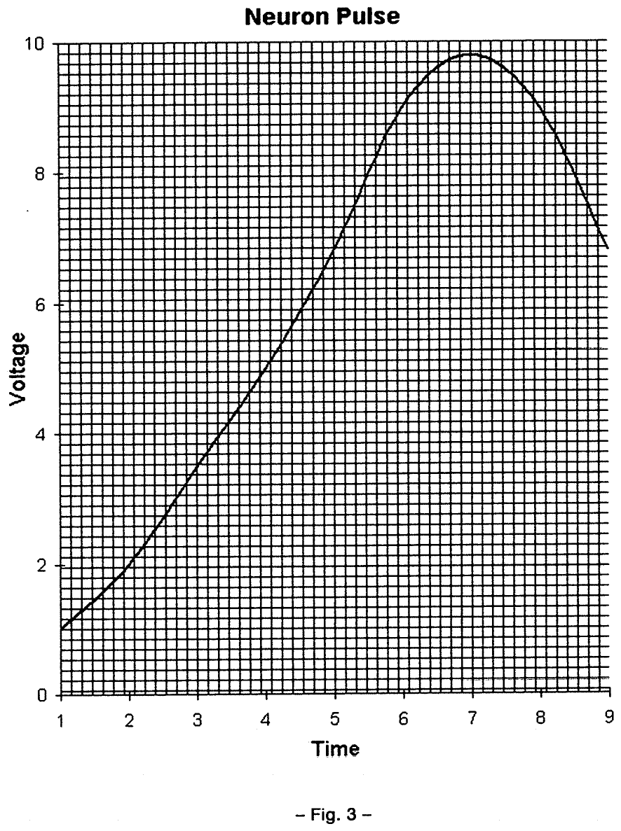

Neurons and transistors alike transmit information as pulses of electromagnetic potential, or “voltage.” Before a neuron can send a pulse, it first must build up the energy for the pulse. FIG. 3 illustrates the time a neuron takes to accumulate this voltage, which is called pulse rise time.

Once the energy in the neuron reaches the “threshold value” necessary to send a pulse (i.e., the top of the curve shown in FIG. 3), a spurt of energy is released from the neuron. This pulse is often called a neuron “spike,” and its voltage is what brainwave measuring devices sense and convert into brainwave frequencies. For example, an average rate of 30 “spikes” per second would be reported by EEG as a brainwave frequency of 30 Hertz.

The “spike” of flowing electrons is transmitted from one neuron to the next one across the synaptic gap via neurotransmitter receptors. The 5-hydroxytryptamine 2A receptors are one such type of receptor.

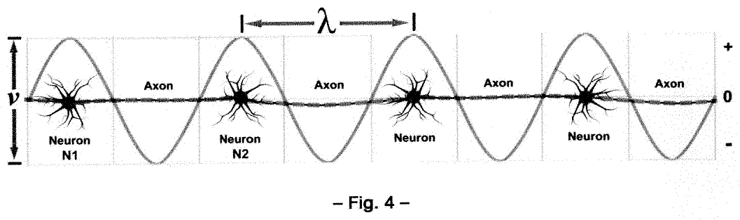

FIG. 4 shows four neurons connected in a series by axons. Each neuron emits a pulse, which collectively form an electromagnetic wave or “neurowave.” The neurowave is shown plotted against voltage grid v.

As illustrated in FIG. 4, the neurowave's wavelength λ is equal to the time between peaks in the wave. This can be expressed mathematically as λ=P+A, where:

λ=Wavelength of neurowave;
P=Neuron pulse rise time; and
A=Axon transmission time.

In FIG. 4, the wave is energized when Neuron N1 fires, then decays over the axon transmission until it is re-energized when the next Neuron N2 fires.

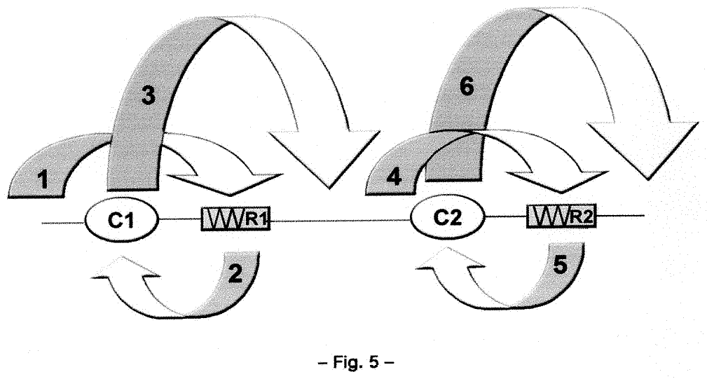

To further clarify how neurons generate electromagnetic waves, consider the neuron's counterpart inside a computer. In electrical engineering, networks of resistors and capacitors are utilized to convey signal pulses comprised of electromagnetic waves. As illustrated in FIG. 5, flowing electrons as shown in arrow 1 encounter a resistor R1 and, as shown in arrow 2, fall back into capacitor C1 behind it, where they are stored. When the capacitor C1 accumulates enough charge to exceed the threshold value of the resistor R1, all the stored electrons in the capacitor flow over the resistance barrier, creating an electromagnetic wave as shown in arrow 3.

Similarly, the neuron acts as both a resistor and a capacitor. As a resistor, it stops the electrons which flow into it from the axon, like a dam halts the flow of water in a river. As a capacitor, it stores and holds the electrons, like a reservoir holds the water behind a dam. The electromagnetic wave which overflows the dam as shown in arrow 3 is the neurowave. The process repeats itself as illustrated in arrows 4, 5 and 6 as the neurowave propagates itself through neurons and axons along the neural pathway.

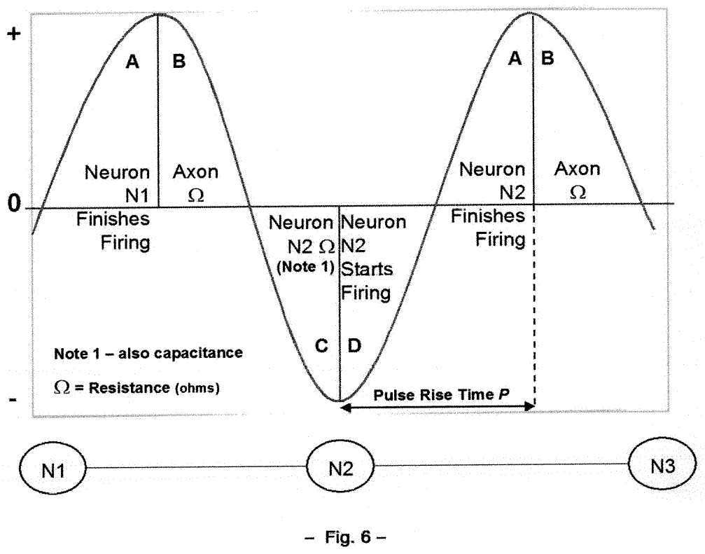

Specifically, the electrical characteristics of the neurowave can be divided into four quadrants: A, B, C, and D, as shown in FIG. 6.

Quadrant A: Neuron N1 releases its pulse signal at the peak of quadrant A. The high voltage at the peak of the wave impels the signal across the axon.
Quadrant B: The signal's voltage diminishes in quadrant B above as it travels across the resistance of the axon.
Quadrant C: Negatively-charged electrons meet Neuron N2's resistance, and gather in the capacitance reservoir of Neuron N2.
Quadrant D: Neuron N2 begins to fire, causing the process to repeat itself.

### 3. Conclusions

Raising neuron resistance decreases brain current density and brainwave activity, as recapped below:

a) Brain current

Brain current can be expressed by

$$Ohm ' s Law I = \frac{E}{R}$$

where:
I=Brain current
E=Brain voltage
R=Resistance of neuron

Hence, raising resistance R decreases brain current I.

b) Brainwave activity

In a neurowave wavelength expressed λ=P+A, where:

λ=Wavelength of neurowave;
P=Neuron pulse rise time; and
A=Axon transmission time:

Assuming fixed axon length, wavelength is a direct function of pulse rise time. Pulse rise time lengthens as neuron resistance rises. Hence, raising neuron resistance increases a neurowave's wavelength, decreasing the number of neuron spikes per unit of time (which collectively comprise brainwave activity).

### 4. Application to Cognitive Enhancement

Recent neuroscience experiments at 15 universities have conclusively demonstrated that reduced brainwave activity is accompanied by higher states of awareness, concentration, focus, mental acuity and cognitive ability. Accordingly, attenuating a subject's brainwave activity will yield a cognitive enhancement.

### 5. Receptor Choice

Numerous neuroscience experiments associate down-regulating the 5-hydroxytryptamine 2A (5-HT2A) receptor with reduced brainwave power and expanded states of cognitive capacity. Accordingly, the 5-HT2A receptor is a prime candidate for use in genetic cognitive engineering.

### III. Methodology

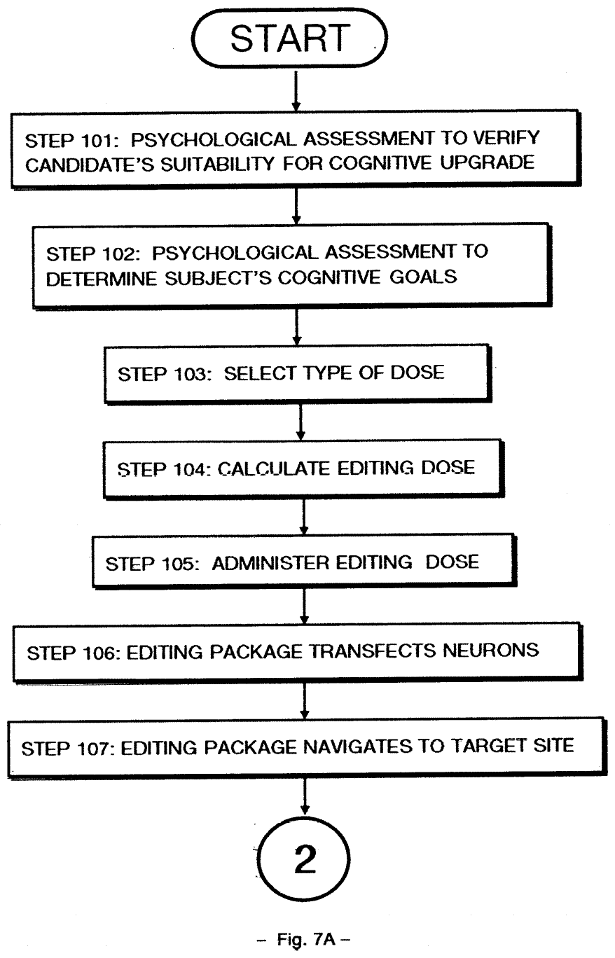

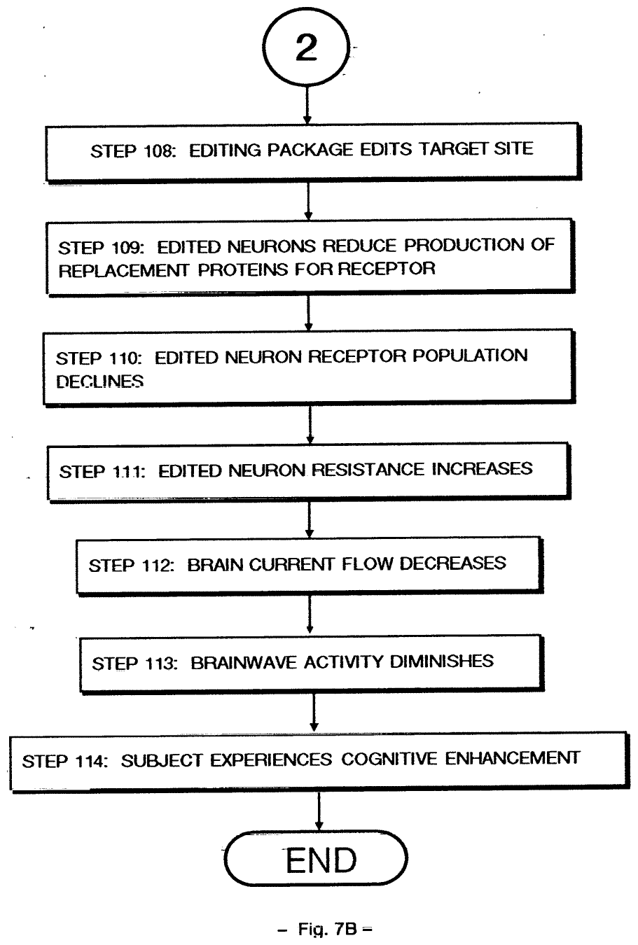

FIGS. 7A and 7B illustrate a method for sustainable human cognitive enhancement. This method employs an RNA-editing ribonuclease complexed with a single guide RNA for lowering the population of 5-hydroxytryptamine 2A receptors in the brain, referred to as an “editing package,” which can be fabricated and manufactured by methods well known in the art. Referring to FIG. 7A:

### Step 101: Psychological Assessment to Verify Candidate's Suitability for Cognitive Upgrade

General-purpose genetic cognitive enhancement is suitable for adults in sound mental and emotional health. The process begins with a psychological assessment to screen out candidates who do not meet this criteria, for example, individuals with alcohol or substance abuse, bipolar disorder, depression, schizophrenia or other psychological conditions or disorders.

The assessment also ensures the candidate is not currently taking any drugs, medications or substances that could interfere with the normal, natural functioning of their brain; for example, alcohol, caffeine, nicotine, cannabis, nootropics, ginseng or other similar substances or herbal preparations.

Candidates who satisfactorily meet the psychological assessment criteria are accepted as subjects for cognitive enhancement.

### Step 102: Psychological Assessment to Determine Subject's Cognitive Goals

The second step is a psychological assessment to ascertain the subject's cognitive enhancement goals. This assessment covers topics such as whether the cognitive upgrade is to be permanent or reversible, and, if reversible, the length of time the upgrade shall have effect.

### Step 103: Select Type of Dose

The type of dose is chosen based on the outcome of the assessment. If the subject's cognitive enhancement is to be reversible after 24 hours, a one-time dose is administered. For the enhancement to persist for longer periods, such as weeks or months, a daily dose is administered.

### Step 104: Calculate Editing Dose

### 1. Background

As shown in Table 1, chemical and transcriptome engineering doses work much differently. A chemical dose's effects occur at the individual receptor level, whereas a transcriptome engineering dose's effects occur at the neuron level. Hence, a chemical dose can affect all, some or none of a neuron's 5-HT2A receptors, whereas a transcriptome dose will affect some or all of a neuron's 5-HT2A receptors.

TABLE 1

| ATTRIBUTE | CHEMICAL DOSE | TRANSCRIPTOME DOSE |
|---|---|---|
| Dose target: | Neuron receptor | Neuron RNA for building receptor |
| Dose disables: | One 5-HT2A | Translation of one gene in one |
| | receptor | neuron having many 5-HT2A |
| | | receptors |
| Amount of | A miniscule | Almost 100%. Transcriptome edits |
| dose that | percent | can be precisely targeted to brain |
| reaches brain: | (e.g., 0.01%) | neurons using navigational guides. |
| Dose is | 5-HT2A receptors | Neurons having between 0 and |
| absorbed by: | on neurons. | 1000+ 5-HT2A receptors |
| Editing | N/A | Current editing efficiency for |
| efficiency | | individual RNA molecules is |
| | | >100%. |

### 2. Formula

A variety of formulas can be developed to calculate dosages based on different subject needs and applications. Given below is a simplified example of a formula for calculating a transcriptome engineering cognitive enhancement dose which is equivalent to a given chemical cognitive enhancement dose which temporarily disables 5-HT2A receptors. Open source neuron simulation models, such as Yale's NEURON model, can be used to calculate precise dosages.

### 1. Receptors Affected Per Chemical Dose (RCD)

Calculate the number of 5-HT2A receptors affected by a known chemical dose (CDR).

a) Known chemical dose=n molecules
b) Approximately y % of dose reaches the brain
c) n molecules x y %=m molecules
d) 1 molecule affects 1 receptor
e) The number of 5-HT2A receptors affected by a chemical dose (RCD)=m receptors.

### 2. Equivalent Number of Neurons (ENN)

Calculate the number of neurons whose total combined 5-HT2A receptor population equals the number of receptors affected by a chemical dose (RCD).

a) Receptors per dendrite=Rd
b) Dendrites per neuron=Dn
c) Receptors per neuron=Rn. Rn=Rd×Dn
d) Average percent of receptors which are 5-HT2A receptors=p %
e) Average number of 5-HT2A receptors per neuron=Rh. Rh=Rn×p %
f) From step 1, receptors affected by chemical dose (RCD)=m receptors.
g) m receptors divided by Rh 5-HT2A receptors per neuron=s neurons
h) The number of neurons whose combined total 5-HT2A receptor population equals the number of receptors affected by a known chemical dose is s neurons. This is the Equivalent Number of Neurons (ENN).

Note: The ENN is used to determine how many neurons to edit. Since editing one neuron's RNA may affect all of a neuron's 5-HT2A receptors, the ENN number takes into account all of each neuron's 5-HT2A receptors. Exceptions to this rule are covered in the next step.

### 3. Half-Life Factor (HLF)

If the half-life of the edited mRNA is any less than the halt-life of the neuron's 5-HT2A receptor proteins, then less than 100% of the neuron's 5-HT2A receptors will degrade during the life of the edited mRNA.

$$Half \text{-} Life Factor ( H L F ) = \frac{Edited mRNA Half \text{-} Life}{Receptor Protein Half \text{-} Life}$$

### 4. Editing Efficiency Factor (EEF)

Include the effects of factors which constrain transcriptome editing efficiency.

a) Transcriptome Editing Efficiency (TEE %) is e % with current technology, meaning that e % of the edits which are absorbed by neurons will be effective.
b) Neurons transfected With Receptor (NWR %): Although the 5-HT2A receptor is widely expressed in the neural cortex, some of the neurons which absorb the transcriptome editing dose will not have the receptor. The Neurons With Receptor (NWR %) factor is g %, meaning that g % of neurons which absorb the transcriptome editing dose possess 5-HT2A receptors.

### c) Editing Efficiency Factor (EFF)=TEE %×NWR %

### 5. Transcriptome Dose

The transcriptome editing dose which is equivalent to the chemical dose is calculated as follows:

$$\frac{\begin{matrix}Equivalent Number of Neurons ( E N N ) \times \\ Half Life Factor ( H L F )\end{matrix}}{Editing Efficiency Factor ( E F F )}$$

### Step 105: Administer Editing Dose

Editing package doses can be administered to subjects via oral, sublingual, or transdermal application or through other methods well known in the art.

### Step 106: Editing Package Transfects Neurons

The guide RNA in the editing package serves as a vector which transfects CNS neurons.

### Step 107: Editing Package Navigates to Target Site

Once inside the cell, the guide RNA in the editing package navigates the package to the target editing site. This can be accomplished with considerable precision using currently-available RNA editing guides such as single-guide RNA (sgRNA).

Five types of editing packages are given here as examples of embodiments of the invention.

1. RNA knockout
2. RNA silencing
3. RNA nucleobase substitutions
4. RNA knock-in
5. Ribosome RNA knockdown

The target editing site for modalities 1 through 4 is the messenger RNA for gene HTR2A. The target editing site for modality 5 is the biogenesis processing site for micro-RNA for gene HTR2A.

Referring to FIG. 7B:

### Step 108: Editing Package Edits Target Site

Once delivered to the target site, the editing package edits the target site as follows:

1. RNA knockout: A catalytically-active RNA-editing ribonuclease, such as Cas 13, is complexed with a single guide RNA for altering RNA nucleotides to repress translation of gene HTR2A.
2. RNA silencing: A catalytically-inactive RNA-editing ribonuclease, such as Cas 13d, is complexed with a single guide RNA for binding to RNA nucleotides to repress translation of gene HTR2A.
3. RNA nucleobase substitutions: A catalytically-inactive RNA-editing ribonuclease complexed with a deaminase enzyme, such as “REPAIR,” is complexed with a single guide RNA to cause RNA nucleobase substitutions which result in translational interference of gene HTR2A.
4. RNA knock-in: An RNA-editing ribonuclease complexed with an RNA-expression inhibiting nucleotide, such as “TUNR,” is complexed with a single guide RNA to alter RNA nucleotides to cause translational interference of gene HTR2A.
5. RNA knockout: A catalytically-active ribonuclease, such as Cas 13, is complexed with a single guide RNA to alter nucleotides in micro-RNA biogenesis sites to repress the expression of gene HTR2A.

### Step 109: Edited Neurons Reduce Production of Replacement Proteins for Receptor

Gene HTR2A supplies neurons with the blueprints for manufacturing the cellular proteins used to build 5-hydroxytryptamine 2A receptors. When this gene's RNA transcripts are knocked out, silenced, altered or inhibited, or their micro-RNA biogenesis sites are knocked down, the neuron makes fewer proteins needed to replace its 5-hydroxytryptamine 2A receptors.

### Step 110: Edited Neuron Receptor Population Declines

There are fifty different types of neuron receptors, and neurons typically contain a mixture of multiple types of receptors. When some of a neuron's 5-hydroxytryptamine 2A receptors are not replaced, its overall number of receptors declines.

### Step 111: Edited Neuron Resistance Increases

A neuron's receptor sites serve as doorways which receive the flow of electrically-charged ions into the neuron. A neuron will fill its cellular reservoir with incoming charged ions more quickly if it has a larger number of receptor sites

TABLE 2

| Receptor | | | Current |
|---|---|---|---|
| Sites | Resistance | Excitability | Flow |
| [symbol] | [symbol] | [symbol] | [symbol] |
| [symbol] | [symbol] | [symbol] | [symbol] |

Referring to Table 2, increasing the number of a neuron's receptor sites adds more channels for incoming ions to flow into, similar to adding more lanes to a freeway. This gives the neuron lower electrical resistance, which makes it more easily excitable.

Conversely, decreasing a neuron's receptor population reduces the number of pipes for incoming ions to flow into, like closing lanes on a freeway. This raises the neuron's electrical resistance, making it harder to excite.

A neuron's resistance can be modified by changing its number of receptor sites. Reducing a neuron's number of receptor sites by removing its 5-HT2A receptors decreases the number of doorways or pipes for electrically-charged ions to flow through, thereby increasing the neuron's resistance. This decelerates the flow of electrons from one neuron to another.

### Step 112: Brain Current Flow Decreases

Raising a neuron's resistance lowers its conductivity. Less-conductive neurons have a lower capacity for carrying the flow of electrical current in the brain.

### Step 113: Brainwave Activity Diminishes

A moving electrical current generates an electromagnetic wave (per Ampere's Law). Flowing electrons in the brain generate brainwaves. When the flowing electrons slow down, so does brainwave activity.

Less-conductive, less-excitable neurons require more time to fill their cellular reservoirs with enough electrically-charged ions to cause them to fire. Hence, they fire less frequently. Lower neuron activity reduces brainwave activity.

### Step 114: Subject Experiences Cognitive Enhancement

Numerous scientific studies have conclusively demonstrated reduced brainwave activity is correlated with higher states of awareness, concentration, focus, mental acuity and cognitive ability. Accordingly, attenuating the subject's brainwave activity will yield a cognitive enhancement.

Although specific embodiments of the invention have been disclosed herein in detail, it is to be understood that this is for the purpose of illustrating the invention, and should not be construed as necessarily limiting the scope of the invention, since it is apparent that many changes can be made to the disclosed methods by those skilled in the art to suit particular applications.

What is claimed is:

1. A reversible method for general-purpose cognitive enhancement in mentally healthy adults comprising administering an RNA-editing ribonuclease complexed with a single guide RNA to lower the population of 5-hydroxytryptamine 2A receptors in the brain.

2. A method as recited in claim 1 where a catalytically-active RNA-editing ribonuclease is complexed with a single guide RNA to lower the population of 5-hydroxytryptamine 2A receptors in the brain by altering RNA nucleotides to repress translation of gene HTR2A into cellular proteins in CNS neurons.

3. A method as recited in claim 1 where a catalytically-inactive RNA-editing ribonuclease is complexed with a single guide RNA to lower the population of 5-hydroxytryptamine 2A receptors in the brain by binding to RNA nucleotides to repress translation of gene HTR2A into cellular proteins CNS neurons.

4. A method as recited in claim 1 where a catalytically-inactive RNA-editing ribonuclease is complexed with a single guide RNA and a deaminase enzyme to lower the population of 5-hydroxytryptamine 2A receptors in the brain by causing RNA nucleobase substitutions which result in translational interference of gene HTR2A in CNS neurons.

5. A method for general-purpose cognitive enhancement in mentally healthy adults comprising administering a RNA-editing ribonuclease complexed with an RNA-expression inhibiting nucleotide and a single guide RNA to lower the population of 5-hydroxytryptamine 2A receptors in the brain by altering RNA nucleotides to cause translational interference of gene HTR2A in CNS neurons.

6. A method for general-purpose cognitive enhancement in mentally healthy adults comprising administering a catalytically-active ribonuclease complexed with a single guide RNA to lower the population of 5-hydroxytryptamine 2A receptors in the brain by altering nucleotides in biogenesis processing sites for micro-RNA used in the translation of gene HTR2A in order to reduce its expression in CNS neurons.
